(19) United States
(12) Patent Application Publication
(10) Pub. No.: US 2023/0298697 A1
(43) Pub. Date: Sep. 21, 2023

(54) DETERMINING VIABILITY AND TREATMENT OF DISEASE AGENTS

(71) Applicant: Institute for Systems Biology, Seattle, WA (US)

(72) Inventors: Nitin BALIGA, Seattle, WA (US); Vivek SRRINIVAS, Karnataka (IN)

(73) Assignee: Institute for Systems Biology, Seattle, WA (US)

(21) Appl. No.: 18/168,056

(22) Filed: Feb. 13, 2023

Related U.S. Application Data
(60) Provisional application No. 63/309,431, filed on Feb. 11, 2022.

(51) Int. Cl. G16B 25/10; G16B 50/20; B01L 3/00; G16H 50/20

(52) U.S. Cl. CPC G16B 25/10; B01L 3/502761; G16B 50/20; G16H 50/20

(57) ABSTRACT

Predicting viability and treatment of disease agents is described herein. In an example, a system accesses a disease agent transcriptome data of a disease agent. The system generates a disease agent viability score by applying a classifier to the disease agent transcriptome. The classifier defines a universal transcriptome signature for a viability of the disease agent in different host-relevant contexts. The system generates a viability state of the disease agent by determining a deviation of the disease agent viability score from a viability threshold of the universal transcriptome signature for viability and determines a treatment recommendation based on the viability state of the disease agent. The system outputs the treatment recommendation.

### CROSS-REFERENCE TO RELATED APPLICATIONS

This application claims the benefit of and priority to U.S. Provisional Application No. 63/309,431, filed on Feb. 11, 2022, which is hereby incorporated by reference in its entirety for all purposes.

### STATEMENT REGARDING FEDERALLY SPONSORED RESEARCH OR DEVELOPMENT

This invention was made with government support under INV-009322 awarded by the Bill and Melinda Gates Foundation and under R01AI128215, R01AI141953, and U19AI135976 awarded by the National Institute of Allergy and Infectious Diseases. The government has certain rights in the invention.

### FIELD

Embodiments relate to generating a treatment recommendation for a disease agent by using a classifier to process disease agent transcriptomes.

### BACKGROUND

The discovery of effective multidrug combinations for treating a disease agent is a challenging endeavor, burdened by the large number of testable drug combinations. For example, a collection of 1,000 drug compounds yields approximately 500,000 pairwise combinations and exponentially larger numbers of higher-order combinations. Multicomponent drug discovery is particularly challenging for some disease agents, such as Mycobacterium tuberculosis, which is a slow-growing pathogen that is capable of generating phenotypically heterogeneous subpopulations. These phenotypically diverse subpopulations allow Mycobacterium tuberculosis, to persist and survive the variable conditions encountered during infection as well as thwart drug treatment. Because of drug-tolerant subpopulations within a host, a large proportion of drug regimens that are effective in killing Mycobacterium tuberculosis in vitro are futile in subjects.

Therefore, it would be advantageous to develop new approaches to reduce the search space and prioritize drug combinations for experimental testing, while also taking into account the host context and different subpopulations of a disease agent.

### SUMMARY

In some embodiments, a computer-implemented method that includes: (a) accessing a disease agent transcriptome of a disease agent; (b) generating a disease agent viability score by applying a classifier to the disease agent transcriptome, the classifier defining a universal transcriptome signature for a viability of the disease agent in a plurality of different host-relevant contexts; (c) generating a viability state of the disease agent by determining a deviation of the disease agent viability score from a viability threshold of the universal transcriptome signature for viability; (d) determining a treatment recommendation based on the viability state of the disease agent; and (e) outputting the treatment recommendation.

The classifier may have been trained using a training data set comprising a plurality of viable disease agent transcriptomes, and the classifier may have been tested on testing data set comprising a first set of untreated disease agent transcriptomes and a second set of treated disease agent transcriptomes. The training data set and the testing data set may have been derived from the disease agent being grown under the plurality of host-relevant contexts with drug treatment and without drug treatment to define the universal transcriptome signature for viability.

The viability threshold may be set as a lower limit of a viable transcriptome space defined by the classifier.

The classifier may be a single-class support vector machine.

The disease agent viability score may be a weighted sum of a plurality gene expression ranks generated by the classifier and rank normalized.

The disease agent may be a cell, and the disease agent transcriptome may be obtainable from the cell.

The disease agent may be Mycobacterium tuberculosis, and a host of the disease agent may be a mammal.

The disease agent transcriptome may comprise a subset of mRNA transcripts produced by primer-directed amplification, and the subset of mRNA transcripts may comprise one or more weighted features selected by bootstrapping and rank ordering based on weights determined by the primer-directed amplification.

The primer-directed amplification may be reverse transcription loop-mediated isothermal amplification (LAMP).

Determining the treatment recommendation may comprise: comparing the viability state of the disease agent to one or more single-drug treatment viability states of the disease agent, the one or more single-drug viability states produced by: (i) generating one or more single-drug treatment viability scores by an application of the classifier to a plurality of single-drug treatment transcriptomes of the disease agent grown under a plurality of single-drug treatment conditions, and (ii) generating the one or more additional viability states by a determination of a deviation of the one or more single-drug treatment viability scores from the viability threshold of the universal transcriptome signature for viability.

Determining the treatment recommendation may further comprise: comparing the viability state of the disease agent and the one or more single-drug viability states of the disease agent with a multi-drug viability state, the multi-drug viability state imputed by an application of the classifier to an average of a plurality of disease agent transcriptomes and one or more single drug treatment transcriptomes.

The average may be a geometric mean.

Determining the treatment recommendation may comprise evaluating an efficacy of a drug treatment for the disease agent.

The method may include facilitating the treatment recommendation for a host of the disease agent.

In some embodiments, a computer-program product is provided that is tangibly embodied in a non-transitory machine-readable storage medium, and that includes instructions configured to cause one or more data processors to perform a set of actions including: (a) accessing a disease agent transcriptome of a disease agent; (b) generating a disease agent viability score by applying a classifier to the disease agent transcriptome, the classifier defining a universal transcriptome signature for viability of the disease agent in a plurality of different host-relevant contexts; (c) generating a viability state of the disease agent by determining a deviation of the disease agent viability score from a viability threshold of the universal transcriptome signature; (d) determining a treatment recommendation for the disease agent based on the viability state of the disease agent; and (e) outputting the treatment recommendation.

Determining the treatment recommendation may comprise: comparing the viability state of the disease agent to one or more single-drug treatment viability states of the disease agent, the one or more single-drug treatment viability states produced by a process comprising an application of the classifier to a plurality of single-drug treatment transcriptomes of the disease agent grown under a plurality of single-drug treatment conditions.

Determining the treatment recommendation further may comprise: comparing the disease agent viability state and the one or more single-drug treatment viability states with a multi-drug treatment viability state.

The multi-drug treatment viability state may be imputed.

The imputed multi-drug treatment viability state may be produced by an imputation comprising an application of the classifier to an average of a plurality of disease agent transcriptomes and one or more single-drug treatment transcriptomes.

In some embodiments, a system is provided that includes: a microfluidic device for receiving a sample of a host subject and producing disease agent transcriptome data of a disease agent from the sample; one or more data processors; and a non-transitory computer readable storage medium containing instructions which, when executed on the one or more data processors, cause the one or more data processors to perform a set of actions including: (a) accessing a disease agent transcriptome of a disease agent; (b) generating a disease agent viability score by applying a classifier to the disease agent transcriptome, the classifier defining a universal transcriptome signature for viability of the disease agent in a plurality of different host-relevant contexts; (c) generating a viability state of the disease agent by determining a deviation of the disease agent viability score from a viability threshold of the universal transcriptome signature; (d) determining a treatment recommendation for the disease agent based on the viability state of the disease agent; and (e) outputting the treatment recommendation.

### BRIEF DESCRIPTION OF THE DRAWINGS

The present disclosure is described in conjunction with the appended figures:

FIG. 1 shows an exemplary computing system for facilitating identification of a treatment recommendation for a disease agent based on disease agent transcriptome data according to some aspects of the present disclosure;

FIG. 2 illustrates an exemplary process of identifying of a treatment recommendation for a disease agent based on disease agent transcriptome data according to some aspects of the present disclosure;

FIG. 3 shows exemplary results of a correlation between a cell viability score and relative colony forming units with and without drug treatment;

FIG. 4 shows exemplary results of classifier-generated cell viability scores for transcriptomes of Mycobacterium tuberculosis;

FIG. 5 shows exemplary results of a comparison of classifier-generated cell viability state with bacteriological assays;

FIG. 6 shows exemplary results of a prediction of drug interaction;

FIG. 7 shows exemplary results of a comparison of models in predicting interaction of 2- and 3-drug combinations;

FIG. 8 shows exemplary results of a correlation between a determined score and fractional inhibitory concentrations for two- and three-drug combinations;

FIG. 9 illustrates an exemplary overview schematic of a classifier framework;

FIG. 10 illustrates exemplary results of iterative training of a classifier; and

FIG. 11 shows exemplary results of cell viability scores accurately predicting bactericidal effects of isoniazid on Mycobacterium tuberculosis in an intracellular environment.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### Overview

Typically, development of treatment regimens for a disease agent relies on growth assays to monitor treatment response. Current methods to monitor treatment response include counting of colony forming units (CFUs) on solid agar plates and measuring the time it takes for a sample in liquid culture to become culture positive for the disease agent, in what is termed time to positivity (TTP) assay. Both CFU counting and TTP have drawbacks including loss of sensitivity, vulnerability to contamination, and lengthy time to measure results. Furthermore, a culture on solid media or in liquid media requires actual growth, which limits the detection of disease-agent subpopulations that may be viable but not actively growing. As such, the process of drug evaluation is slow and inefficient owing to the slow growth rates of target cells in many cases, the complexity of performing assays, and the context-dependent variability in drug sensitivity.

Instead, profiling 16S ribosomal ribonucleic acid (RNA) as a proxy for the load of the disease agent in sputum may be a more sensitive technique that addresses the shortcomings of growth-based assays. Information in RNA can be amplified using technologies such as probe capture and polymerase chain reaction (PCR) to develop highly-sensitive methods for investigating drug response of the disease agent, especially from subject samples. These methods may use disease agent transcriptomes obtainable from a disease agent to predict a viability of the disease agent.

Some embodiments relate to using disease agent transcriptome data of a disease agent to determine a viability of the disease agent in different host-relevant contexts. The viability of the disease agent may be used to determine a treatment recommendation for the disease agent. Determining the treatment recommendation may be determined by screening for the presence or absence of the disease agent, evaluating drug response and multidrug interactions, or identifying a treatment regimen of one or more drugs.

One embodiment provides a method for predicting a disease agent viability score for a disease agent and for determining a treatment recommendation for the disease agent based on the disease agent viability score. The method involves accessing a disease agent transcriptome of a disease agent (e.g., bacteria, virus, cancer cells, etc.). A classifier is applied to the disease agent transcriptome to generate a disease agent viability score for the disease agent. The classifier+ defines a universal transcriptome signature for a viability of the disease agent in different host-relevant contexts that mimic one or more physiological attributes of a host of the disease agent. The universal transcriptome signature may represent signature of a transcriptome of the disease agent when not treated with drugs. A disease agent viability state for the disease agent is determined based on a deviation of the disease agent viability score from a viability threshold, which may be set based on a determination of a viable transcriptome space by the classifier. A treatment recommendation for the disease agent is determined based on the disease agent viability state. For instance, the classifier may also determine single drug treatment viability scores of the disease agent grown under single-drug conditions. The disease agent viability state of the disease agent may be compared to the single drug treatment viability states to determine an efficacy of the drug on the viability of the treatment agent. The comparison may include generating one or more single-drug treatment viability scores by an application of the classifier to a plurality of single-drug treatment transcriptomes of the disease agent grown under a plurality of single-drug treatment conditions, and generating the one or more additional viability states by a determination of a deviation of the one or more single-drug treatment viability scores from the viability threshold of the universal transcriptome signature for viability. Based on the comparison, the treatment recommendation can be determined. In addition, multidrug combinations may also be evaluated to determine whether the treatment recommendation should include a two- or three- drug treatment regimen. The treatment recommendation is output and the recommended therapy can be facilitated for the host of the disease agent. This approach may be advantageous since the viability scores accurately reflect drug response and drug interaction in diverse contexts while avoiding the slow and inefficient process of drug evaluation typical of laboratory assays.

### Definitions

“Disease agent” refers to an infectious agent such as a virus, bacteria, or fungus that is capable of spreading a disease to, or causing a disease in a host animal or human being, or a disease cell such as a cell infected with the disease agent or a cancer cell capable of spreading or causing a disease in a host animal or human being.

“Disease agent transcriptome” refers to the set of all RNA transcripts, including coding and non-coding, of a disease agent, or a subset of the RNA transcripts, such as a curated subset of RNA transcripts defining specific genes whose expression levels are diagnostic of the viability state of the disease agent.

“Host-relevant contexts” refers to conditions that mimic the disease agent growing under, or isolated from, physiologically relevant conditions and/or locations of a host of the disease agent, such as ex vivo culturing conditions that mimic the disease agent in a non-human animal or human host of the disease agent.

### Systems and Methods for Statin Therapy Intensity Prediction

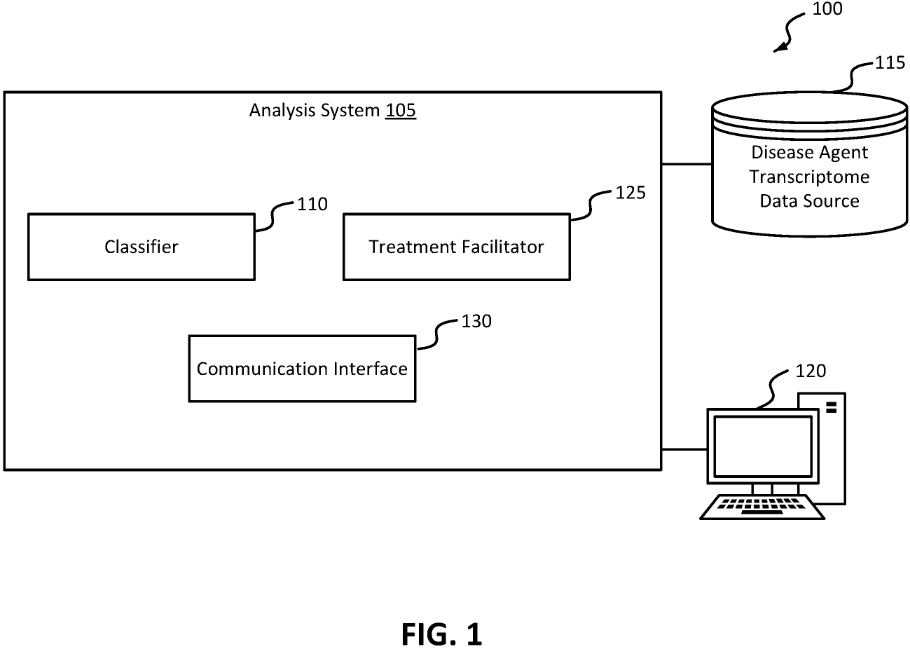

FIG. 1 shows an exemplary computing system 100 for facilitating identification of a treatment recommendation for a disease agent based on disease agent transcriptome data. The computing system 100 can include an analysis system 105 to execute a classifier 110 for determining a disease agent viability score of the disease agent in host-relevant contexts. The classifier 110 can define a universal transcriptome signature for a viability of the disease agent in different host-relevant contexts. The classifier 110 may include one or more machine-learning algorithms. Examples of the machine-learning model include a support vector machine, a decision tree, k-nearest neighbor model, a logistic regression model, etc. The machine-learning model may be trained and/or used to (for example) predict the disease agent viability score from which a viability state and a treatment recommendation for a disease agent can be determined.

In some instances, the classifier 110 may be trained using training data of one or more training data sets. Each training data set of the can include various viable disease agent transcriptomes. Each viable disease agent transcriptome in a first subset of the set of training data may be associated with being grown in optimal growth conditions (e.g., mid-log phase of growth in 7H9-rich media, incubated at 37° C. with aeration) and each subject in a second subset of the set of training data may be associated with a culture of the disease agent being treated with more than one minimum inhibitory concentration 50 (MIC50) drug for greater than a specified period of time (e.g., 12 hours). The training data may have been collected (for example) from one or more data sources, such as a disease agent transcriptome data source 115 that stores disease agent transcriptome data for disease agents.

The computing system 100 can map the training data in the first subset to a “viable” label and the training data in the second subset to a “non-viable” label. Remaining training data can be mapped to an “unclassified” label. Mapping data may be stored in a mapping data store (not shown). The mapping data may identify each disease agent transcriptome that is mapped to each of the labels. In some instances, labels associated with the training data may have been received or may be derived from data received from one or more provider systems 120, each of which may be associated with (for example) a user, nurse, treatment facility, etc. associated with a particular subject.

The analysis system 105 can use the mappings of the training data to train the classifier 110. More specifically, the analysis system 105 can access an architecture of a model, define (fixed) hyperparameters for the model (which are parameters that influence the learning rate, size, and complexity of the model, etc.), and train the model such that a set of parameters are learned. More specifically, the set of parameters may be learned by identifying parameter values that are associated with a low or lowest loss, cost or error generated by comparing predicted outputs (obtained using given parameter values) with actual outputs.

The training may, but need not, involve performing rank normalization on the viable disease agent transcriptomes. The rank normalized viable disease agent transcriptomes can be input along with the corresponding labels to the classifier 110. The training can involve iterations of training the classifier 110 on the viable disease agent transcriptomes labelled as “viable” and then calculating an accuracy of the classifier 110 using a testing data set. The testing data set can include untreated disease agent transcriptomes and treated disease agent transcriptomes. The testing data set may be derived from growing the disease agent under host-relevant contexts with drug treatment and without drug treatment. The accuracy can then be assessed and parameters of the classifier 110 may be adjusted. The training may additionally involve predicting the viability of viable disease agent transcriptomes labelled as “unclassified” and moving the viable disease agent transcriptomes to the first subset associated with the “viable” label. The iterative process may be stopped when the accuracy of the classifier 110 drops below an accuracy threshold (e.g., 85%) or when no new viable disease agent transcriptomes from the “unclassified” set are found to be viable.

Once trained, the classifier 110 can use the architecture and learned parameters to process non-training data and generate a result. For example, classifier 110 may access an input data set that includes disease agent transcriptome data for a disease agent. The disease agent may be a virus, bacteria, or cancer cell in a host. The disease agent transcriptome is obtainable from the disease agent. The disease agent transcriptome may be accessed from the disease agent transcriptome data source 115 or may be received from the provider system 120. For instance, the provider system 120 may include or access a microfluidic device that receives a sample (e.g., broth culture, macrophage infection, or sputum) of a host and produces disease agent transcriptome data from the sample. In some examples, the disease agent transcriptome includes a subset of mRNA transcripts produced by primer-directed amplification of the disease agent. The subset of mRNA transcripts may include weighted features selected by bootstrapping and rank ordering based on weights determined from the primer-directed amplification. An example of primer-directed amplification is reverse transcription loop-mediated isothermal amplification (LAMP).

The input data set can be fed into the classifier 110 having an architecture (e.g., single-class support vector machine) used during training and configured with learned parameters. The classifier 110 can define a universal transcriptome signature for a viability of the disease agent in host-relevant contexts and output a prediction of a disease agent viability score for the disease agent. The host-relevant contexts can be conditions that mimic physiological attributes (e.g., temperature, pH, pressure, etc.) and/or location attributes of a host of the disease agent.

The prediction of the disease agent viability score of the disease agent can be used by the analysis system 105 to determine a viability state for the disease agent. The viability score can represent an empirical distance from the viable class determined by the classifier 110 and can be indicative of efficacious drug treatment. The viability score may be based on a deviation of the disease agent viability score from a viability threshold of the universal transcriptome signature. In general, the disease agent viability score is a weighted sum of gene expression ranks produced by the classifier 110, and rank normalized. If the disease agent is a cell, the disease agent viability score may be a cell viability score (CVS), where the cell is infected with the disease agent or is the disease agent. The viability threshold may be set as the lower limit of the classifier-defined viable transcriptome space. For instance, for Mycobacterium tuberculosis, the viability threshold may be -3.5e¹⁰, below which a CVS indicates a viability state of nonviable Mycobacterium tuberculosis. The viability state may be represented qualitatively as “viable” or “non-viable”, or qualitatively as a value between 0 and 1, where 0 corresponds to “non-viable” and 1 corresponds to “viable”. Other representations of the viability state are also possible.

The analysis system 105 may use the viability state to determine a treatment recommendation for the disease agent. Determining the treatment recommendation may involve evaluating or predicting efficacy and/or response of single and multi-drug treatment regimens and facilitating treatment of a host subject based on the evaluation or prediction. Additionally or alternatively, determining the treatment recommendation may involve screening for the presence or absence of the disease agent and evaluating drug response and multidrug interactions.

The analysis system 105 may determine the treatment recommendation by comparing the disease agent viability state of the disease agent to one or more single drug treatment viability states for the disease agent. The one or more single drug treatment viability scores can be generated by applying the classifier 110 to single drug treatment transcriptomes of the disease agent grown under single drug treatment conditions. The one or more single drug treatment viability states can be generated by a determining a deviation of the one or more single-drug treatment viability scores from the viability threshold of the universal transcriptome signature for viability. Examples of drugs include bedaquiline, clofazimine, isoniazid, linezolid, moxifloxacin, pretomanid, and rifampicin. In some embodiments, the comparison is by rank normalization. As an example, the analysis system 105 may determine that the disease agent viability score is greater than a single drug treatment viability score associated with growing the disease agent with bedaquiline, indicating that viability of the disease agent decreases with bedaquiline. So, the treatment recommendation may involve treating the host of the disease agent with bedaquiline. As another example the analysis system 105 may determine that the viability state is nonviable for moxifloxacin and is viable for isoniazid, so the treatment recommendation can involve treating the host of the disease agent with moxifloxacin.

In certain embodiments, the analysis system 105 may determine drug interactions and determine the treatment recommendation based on the drug interactions. For example, the analysis system 105 may compare the disease agent viability state and single drug treatment viability states with a multi-drug treatment viability state. The multi-drug treatment viability score may be imputed by an application of the classifier 110 on an average of disease agent transcriptomes and the single drug treatment transcriptomes for the disease agent. The multi-drug viability state can be determined based on a deviation of the multi-drug viability score from the viability threshold of the universal transcriptome signature. In an example, the average of the disease agent transcriptomes may be determined as the geometric mean. The synergy or antagonism of a drug combination may be predicted based on calculating a ratio of the predicted viability score from the classifier 110 to an expected viability score corresponding the average of disease agent viability scores from respective single-drug treatments. The analysis system 105 may predict synergistic, additive, or antagonistic drug interactions by comparing an average of the single drug treatment viability states to the imputed multi-drug treatment viability state. That is, if the imputed multi-drug viability score for two drugs is greater than the average of the single drug treatment viability scores for the two drugs, the analysis system 105 may determine that the drugs are synergistic in treating the disease agent. Alternatively, if the imputed multi-drug viability score for two drugs is less than the average of the single drug treatment viability scores for the two drugs, the analysis system 105 may determine that the drugs are antagonistic in treating the disease agent. The treatment recommendation may involve treating the host with a treatment regimen of two or more drugs based on the determined drug interactions.

In some instances, personalized drug treatments can be recommended for a subject. For example, the disease agent can be isolated from a subject (or a pre-clinical mouse or non-human primate model) and exposed ex vivo to a panel of drugs (one-at-a-time), followed by isolation of the disease agent transcriptome or a subset thereof and calculating the disease agent viability scores. Effective single or multi-drug combinations can then be determined from the viability scores.

The analysis system 105 can output the treatment recommendation. A therapy facilitator 125 of the analysis system 105 can then facilitate a treatment for the host in accordance with the treatment recommendation. Facilitating the treatment may involve outputting a recommendation for providing a drug to the host according to the treatment recommendation. The recommendation can indicate a dosage for each drug based on the treatment recommendation. The recommendation may additionally include information that is indicative as to why the recommendation is provided. For instance, the information may indicate the disease agent viability scores that contributed to the recommendation.

A communication interface 130 can collect results and communicate the result(s) (or a processed version thereof) to the provider system 120 (e.g., associated with care provider of the subject), or another system. For example, communication interface 130 may generate and output an indication of the treatment recommendation. The recommendation may then be presented and/or transmitted, which may facilitate a display of the treatment recommendation, for example on a display of a computing device.

A particular example relates to using transcriptomes to predict Mycobacterium tuberculosis’ response to drug treatment and classifying two- and three- drug combinations based on a likelihood of synergistic or antagonistic action on Mycobacterium tuberculosis. In this example, Mycobacterium tuberculosis is the disease agent. For example, the classifier 110 can include a first machine learning algorithm, which may be referred to as drug response assayer (DRonA), that was trained and tested on transcriptomes of Mycobacterium tuberculosis cultured under diverse conditions (e.g., with and without perturbation) to detect a gene signature for loss of Mycobacterium tuberculosis viability. Using drug-induced transcriptional changes, DRonA can calculate the cell viability score, corresponding to the disease agent viability score, which distinguishes the extent of a drug’s bacteriostatic or bactericidal activity on Mycobacterium tuberculosis.

In addition, disease agent transcriptomes from single-drug treatment can be used to predict the interaction of drugs in combination. Using the ratio of an expected disease agent viability score (e.g., based on the CVS of individual drugs) and a predicted disease agent viability score (e.g., based on an inferred multi-drug transcriptome generated from single-drug transcriptomes) calculated by DRonA, a second machine learning algorithm of the classifier 110, referred to as “MLSynergy”, can distinguish between synergistic and antagonistic combinations of drugs. An output score from MLSynergy less than 1 may indicate that the drug interaction is synergistic while an output score greater than 1 indicates an antagonistic drug interaction.

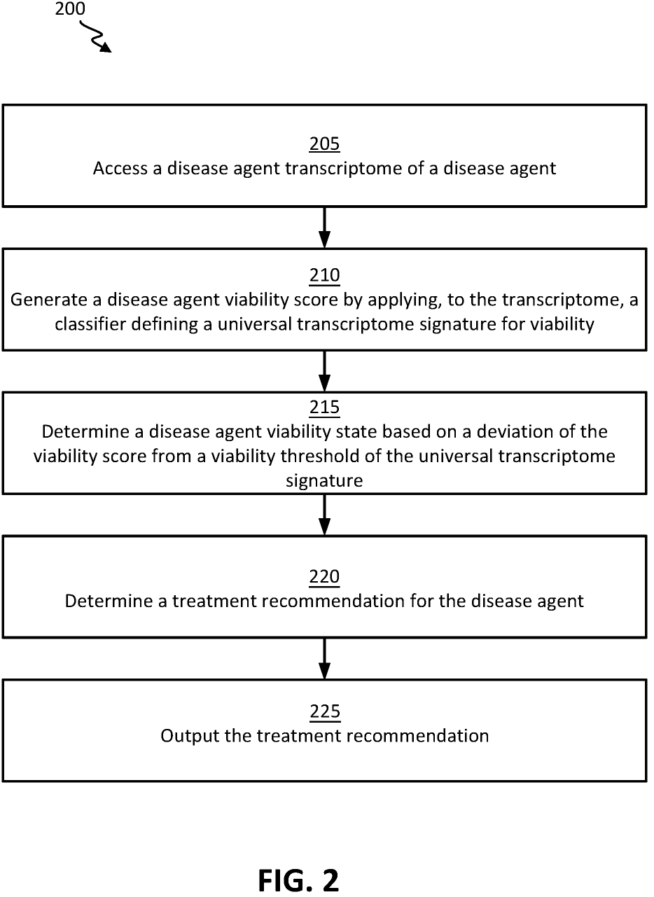

FIG. 2 illustrates an exemplary process 200 of identifying of a treatment recommendation for a disease agent based on disease agent transcriptome data. At block 205, a disease agent transcriptome of a disease agent is accessed. The disease agent may be pathogenic bacteria cells, cancerous cells, and the like. The disease agent transcriptome is obtainable from the disease agent. A microfluidic device can receive a sample (e.g., broth culture, macrophage infection, or sputum) of a host and produce the disease agent transcriptome from the sample. The disease agent transcriptome may be a subset of mRNA transcripts produced by primer-directed amplification of the disease agent.

At block 210, a disease agent viability score is generated by applying, to the transcriptome, a classifier defining a universal transcriptome signature for viability. The viability may be a viability of the disease agent in host-relevant contexts. The host relevant-contexts can represent conditions that mimic physiological attributes of the host. For example, if the host of the disease agent is a human, the host-relevant contexts may mimic temperatures (e.g., 35° C.-39° C.), pH (e.g., 7.35-7.45), pressures, concentrations, etc. of human body. The disease agent transcriptome can be input into a classifier that defines the universal transcriptome signature for viability. The classifier may be a machine-learning model trained to predict the disease agent viability score.

At block 215, a disease agent viability state is determined based on a deviation of the viability score from a viability threshold of the universal transcriptome signature (e.g., 3.5e¹⁰ for Mycobacterium tuberculosis). The deviation can represent an empirical distance from the viable class determined by the classifier. As an example, the disease agent viability score may be a weighted sum of gene expression ranks produced by the classifier and rank normalized. The viability threshold may be set as the lower limit of a viable transcriptome space defined by the classifier.

At block 220, a treatment recommendation for the disease agent is determined. The treatment recommendation may be determined based on the disease agent viability score or the viability state. For instance, if the disease agent viability score is below a threshold or the viability state is nonviable, the treatment recommendation may be to perform no action. Alternatively, if the disease agent viability score is above a threshold or the viability state is viable, the efficacy of one or more drugs on the viability of the disease agent may be evaluated to determine a drug treatment regimen. To determine the efficacy of a single drug, the disease agent viability state of the disease agent may be compared to one or more single drug treatment viability states for the disease agent. The one or more single drug treatment viability scores can be generated by applying the classifier to single drug treatment transcriptomes of the disease agent grown under single drug treatment conditions. The one or more single drug viability states can be determined from a deviation of the one or more single drug treatment viability scores from the viability threshold of the universal transcriptome signature for viability. The drugs may include bedaquiline, clofazimine, isoniazid, linezolid, moxifloxacin, pretomanid, and rifampicin. As an example, it may be determined that the single drug treatment viability score associated with pretomanid is higher than the single drug treatment viability score for rifampicin, indicating that viability of the disease agent is less when treated with rifampicin than with pretomanid. So, the treatment recommendation may involve treating the host of the disease agent with rifampicin.

To determine the efficacy of multiple drugs on the viability of the disease agent, drug interactions can be identified and the treatment recommendation can be based on the drug interactions. For example, the viability state of the disease agent and the one or more single drug viability states may be compared with a multi-drug treatment viability state that is imputed by an application of the classifier on an average of disease agent transcriptomes and the single drug treatment transcriptomes for the disease agent. Drug interactions can then be predicted as being synergistic, additive, or antagonistic by comparing an average of the single drug treatment viability states to the imputed multi-drug treatment viability state. The treatment recommendation may involve treating the host with a treatment regimen of two or more drugs based on the determined drug interactions.

At block 225, the treatment recommendation is output. The treatment recommendation may be output to a computing device associated with a clinician of the host such that the clinician can prescribe the treatment recommendation for the host. In addition, a dosage and drug treatment regimen for the host may be determined based on the treatment recommendation. An indication of the dosage and the drug treatment regimen can be provided to a provider system so that the appropriate drug can be provided to the host.

FIG. 2 shows one exemplary process for predicting a treatment recommendation from disease agent transcriptome data. Other examples can include more steps, fewer steps, different steps, or a different order of steps.

### EXAMPLES

The following examples are provided to illustrate certain particular features and/or embodiments. These examples should not be construed to limit the disclosure to the particular features or embodiments described.

### Bacterial Strains and Growth Conditions

The Mycobacterium tuberculosis strain used in the study was H37Rv. Mycobacterium tuberculosis cells were cultured in standard 7H9-rich media consisting of 7H9 broth with 0.05% Tween-80, 0.2% glycerol, and 10% Middlebrook ADC. Frozen 1 mL stocks of Mycobacterium tuberculosis cells were added to 7H9 medium and grown with mild agitation in a 37° C. incubator until the culture reached an OD600 of approximately 0.4-0.8. The cells were then diluted to OD600 of 0.05 and added to 7H9-rich medium containing drugs at the predetermined amounts.

### Minimum Inhibitory Concentration 50 (MIC50) Determination

10 mM working concentrations of drugs considered in the study were made with a suitable vehicle depending on drug solubility (e.g., water, DMSO, or methanol). The 10 mM working concentrations of drugs were diluted in a two-fold dilution series for 11 concentrations in 96-well plates. Each treatment series contained an untreated well as a control. Mycobacterium tuberculosis H37Rv cultures were added to the wells and the plates were incubated at 37° C. Growth in cultures were measured as OD600 at 0 and 72 hours of incubation. All MIC50 determinations were performed in biological triplicate. Growth inhibition was determined by subtracting the initial reads from the final reads and then normalizing the data to no drug controls. Growth inhibition was fit to a sigmoidal curve and MIC50 was calculated for each drug, as shown in Table 1.

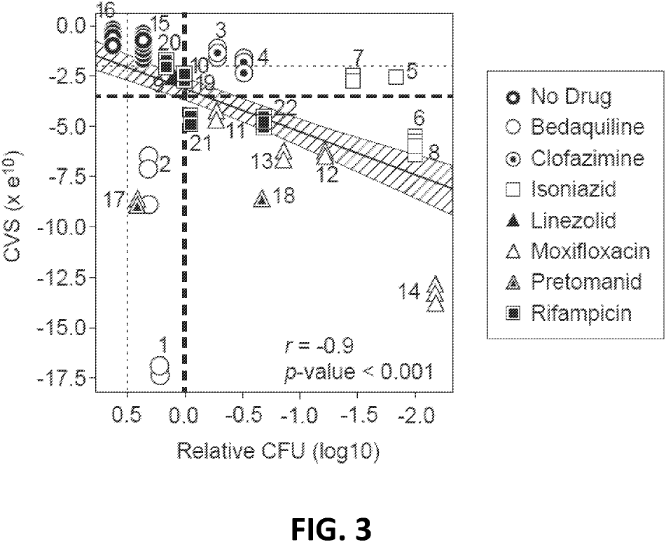

FIG. 3 shows the correlation between cell viability scores (CVS) and relative colony forming units (CFU) with and without drug treatment. Relative CFU was calculated in relation to 0 hours (e.g., prior to drug or vehicle control treatment). Numbers associated with the points indicate specific drug treatment time and concentrations found in Table 1. Relative CFUs for the treatments in Table 1 were calculated with time-kill assay and are given in Table 2. The solid line denotes the Pearson’s correlation between CVS and relative CFU. Significance was calculated as the average correlation coefficient, r, from 100 iterations performed with 70% randomly selected data. The dotted line denotes 50% growth inhibition from drug treatments and its corresponding CVS threshold (-2.25e¹⁰). The dashed line indicates bactericidal activity and its corresponding CVS threshold (-3.5e¹⁰).

TABLE 1

MIC50 calculations for each drug

| Drug | Low concentration (µg/ml) | High concentration (µg/ml) | MIC50 (µg/ml) | Related to FIG. 3 (number legend) |
|---|---|---|---|---|
| Bedaquiline | 5.75 | 11.5 | 1.61 | (1) 5.75 µg/ml, 24 h |
| | | | | (2) 5.75 µg/ml, 72 h |
| Clofazimine | 0.73 | 3.65 | 1.17 | (3) 0.73 µg/ml, 24 h |
| | | | | (4) 0.73 µg/ml, 72 h |
| Isoniazid | 0.36 | 1.8 | 0.2 | (5) 0.36 µg/ml, 24 h |
| | | | | (6) 0.36 µg/ml, 72 h |
| | | | | (7) 1.8 µg/ml, 24 h |
| | | | | (8) 1.8 µg/ml, 72 h |
| Linezolid | 0.84 | 4.2 | 1.13 | (9) 0.84 µg/ml, 24 h |
| | | | | (10) 0.84 µg/ml, 72 h |
| Moxifloxacin | 0.12 | 0.3 | 0.07 | (11) 0.12 µg/ml, 24 h |
| | | | | (12) 0.12 µg/ml, 72 h |
| | | | | (13) 0.3 µg/ml, 24 h |
| | | | | (14) 0.3 µg/ml, 72 h |
| No drug | NA | NA | NA | (15) NA, 24 h |
| | | | | (16) NA, 72 h |
| Pretomanid | 0.7 | 3.5 | 0.15 | (17) 0.70 µg/ml, 24 h |
| | | | | (18) 0.70 µg/ml, 72 h |
| Rifampicin | 0.008 | 0.02 | 0.02 | (19) 0.008 µg/ml, 24 h |
| | | | | (20) 0.008 µg/ml, 72 h |
| | | | | (21) 0.02 µg/ml, 24 h |
| | | | | (22) 0.02 µg/ml, 72 h |

Drug concentrations used for transcriptome profiles generated in this study. The low (bacteriostatic) and high (bactericidal) drug concentrations were selected based on time-kill assays. The MIC50 determination for drugs used in this study, related to Minimum Inhibitory Concentration 50 (MIC50) determination section in STAR methods. The number legend for FIG. 3 , drug treatment concentrations and time used to compare the DRonA generated CVS and relative CFUs.

### Time-Kill Assays

Using growth conditions described above, cells were diluted into 7H9-rich media containing drugs at predetermined amounts, along with vehicle controls (Table 1). Samples were taken after 0, 24 and 72 hours, serially diluted and plated on 7H10 agar plates. All time-kill assays were performed in biological triplicate. Relative colony forming units (CFUs) were calculated as log 10 ratio of CFUs/ml of culture observed at start of treatment (T0) and after drug treatment.

TABLE 2

Relative CFUs from single drug treated time kill curves of Mycobacterium tuberculosis cultures related to FIG. 3 . Relative CFU was measured as a ratio between the CFUs observed at start of the treatment (h0) vs. CFUs observed post treatment.

| Sample # | Drug | Concentration (µg/ml) | CFUs /ml (10⁶) | | | | Relative CFUs (log10) |
|---|---|---|---|---|---|---|---|
| | | | h0 | h24 | h72 | h24 | h72 |
| 2 | BDQ | 1.15 | 61 | 91 | 352 | 0.174 | 0.761 |
| 3 | BDQ | 1.15 | 69 | 96 | 362 | 0.143 | 0.72 |
| 4 | BDQ | 1.15 | 72 | 101 | 346 | 0.147 | 0.682 |
| 5 | BDQ | 5.75 | 55 | 97 | 115 | 0.246 | 0.32 |
| 6 | BDQ | 5.75 | 53 | 81 | 132 | 0.184 | 0.396 |
| 7 | BDQ | 5.75 | 61 | 100 | 96 | 0.215 | 0.197 |
| 18 | CFZ | 0.0728 | 148 | 124 | 207 | -0.077 | 0.146 |
| 19 | CFZ | 0.0728 | 239 | 116 | 176 | -0.314 | -0.133 |
| 20 | CFZ | 0.0728 | 141 | 112 | 192 | -0.1 | 0.134 |
| 21 | CFZ | 0.728 | 158 | 87 | 41 | -0.259 | -0.586 |
| 22 | CFZ | 0.728 | 182 | 69 | 26 | -0.421 | -0.845 |
| 23 | CFZ | 0.728 | 101 | 67 | 52 | -0.178 | -0.288 |
| 36 | INH | 0.018 | 49 | 182 | 184 | 0.57 | 0.575 |
| 37 | INH | 0.018 | 91 | 160 | 194 | 0.245 | 0.329 |
| 38 | INH | 0.018 | 132 | 171 | 203 | 0.112 | 0.187 |
| 39 | INH | 0.18 | 93 | 60 | 78 | -0.19 | -0.076 |
| 40 | INH | 0.18 | 77 | 90 | 97 | 0.068 | 0.1 |
| 41 | INH | 0.18 | 70 | 60 | 70 | -0.067 | 0 |
| 42 | INH | 0.36 | 159 | 1 | 0.1 | -2.201 | -3.201 |
| 43 | INH | 0.36 | 180 | 4 | 0.1 | -1.653 | -3.255 |
| 44 | INH | 0.36 | 187 | 3 | 0.1 | -1.795 | -3.272 |
| 45 | INH | 1.8 | 77 | 1 | 0.1 | -1.886 | -2.886 |
| 46 | INH | 1.8 | 68 | 3 | 0.1 | -1.355 | -2.833 |
| 47 | INH | 1.8 | 87 | 4 | 0.1 | -1.337 | -2.94 |
| 63 | LZD | 0.0844 | 81 | 135 | 249 | 0.222 | 0.488 |
| 64 | LZD | 0.0844 | 100 | 147 | 245 | 0.167 | 0.389 |
| 65 | LZD | 0.0844 | 98 | 127 | 239 | 0.113 | 0.387 |
| 66 | LZD | 0.844 | 61 | 63 | 71 | 0.014 | 0.066 |
| 67 | LZD | 0.844 | 68 | 74 | 86 | 0.037 | 0.102 |
| 68 | LZD | 0.844 | 60 | 92 | 72 | 0.186 | 0.079 |
| 78 | MXF | 0.075 | 69 | 90 | 159 | 0.115 | 0.363 |
| 79 | MXF | 0.075 | 53 | 88 | 165 | 0.22 | 0.493 |
| 80 | MXF | 0.075 | 72 | 80 | 147 | 0.046 | 0.31 |
| 81 | MXF | 0.3 | 43 | 4 | 0.1 | -1.031 | -2.633 |
| 82 | MXF | 0.3 | 50 | 9 | 0.1 | -0.745 | -2.699 |

### Collection, RNA Extraction, and Analysis of Single-Drug Transcriptomes

Using growth conditions described above, cells were diluted into 7H9-rich media containing drugs at predetermined amounts, along with vehicle controls (Table 1 and Table 3). Samples, in biological triplicates, were collected after 24 and 72 hours. Samples were centrifuged at high speed for 5 min, supernatant was discarded, and cell pellet was immediately flash frozen in liquid nitrogen. Cell pellets were stored at -80° C. until bead beating in a FastPrep 120 homogenizer and RNA extraction was performed. Total RNA was depleted of ribosomal RNA using the Ribo-Zero Bacteria rRNA Removal Kit. Quality and purity of the mRNA was determined with a 2100 Bioanalyzer. Sequencing libraries were prepared with TrueSeq Stranded mRNA HT library preparation kit. All samples were sequenced on the NextSeq sequencing instrument in a high output 150 v2 flow cell. Paired-end 75 bp reads were checked for technical artifacts using Illumina default quality filtering steps. Raw FASTQ read data were processed using the R package DuffyNGS. Read counts were further analyzed with Kallisto and RPKM values were calculated.

TABLE 3

Treatments used to generate the transcriptomes used to test DRonA and predict drug interactions with MLSynergy, related to FIGS. 3-6

| Drug | Concentration (µg/ml) | Treatment time (hours) | Growth context | Replicates | Study |
|---|---|---|---|---|---|
| BDQ | 11.5 | 72 | Broth | 3 | This study |
| CFZ | 3.65 | 72 | Broth | 3 | This study |
| INH | 1.8 | 72 | Broth | 3 | This study |
| LZD | 4.2 | 72 | Broth | 3 | This study |
| MXF | 0.3 | 72 | Broth | 3 | This study |
| PA824 | 0.7 | 72 | Broth | 3 | This study |
| POA | 3.5 | 72 | Broth | 3 | This |
| | | | | | study |
| RIF | 0.02 | 72 | Broth | 3 | This study |
| No drug (Lag phase) | 0 | 0 | Broth | 12 | This study |
| No drug (Early log phase) | 0 | 0 | Broth | 12 | This study |
| No drug (Log phase) | 0 | 0 | Broth | 12 | This study |
| No drug (Stationary phase) | 0 | 0 | Broth | 12 | This study |
| EMB | 12 | 24 | Broth | 3 | Liu et al., 2016 |
| INH | 0.4 | 24 | Broth | 6 | Liu et al., 2016 |
| POA | 200 | 24 | Broth | 3 | Liu et al., 2016 |
| RIF | 0.4 | 24 | Broth | 2 | Liu et al., 2016 |
| EMB | 12 | 24 | Intra-macrophage | 3 | Liu et al., 2016 |
| INH | 0.4 | 24 | Intra-macrophage | 8 | Liu et al., 2016 |
| POA | 200 | 24 | Intra-macrophage | 3 | Liu et al., 2016 |
| RIF | 0.4 | 24 | Intra-macrophage | 3 | Liu et al., 2016 |
| No drug (Inf-g) | 0 | 24 | Intra-macrophage | 2 | Liu et al., 2016 |
| No drug (PBS) | 0 | 24 | Intra-macrophage | 4 | Liu et al., 2016 |
| No drug (RAP) | 0 | 24 | Intra-macrophage | 2 | Liu et al., 2016 |

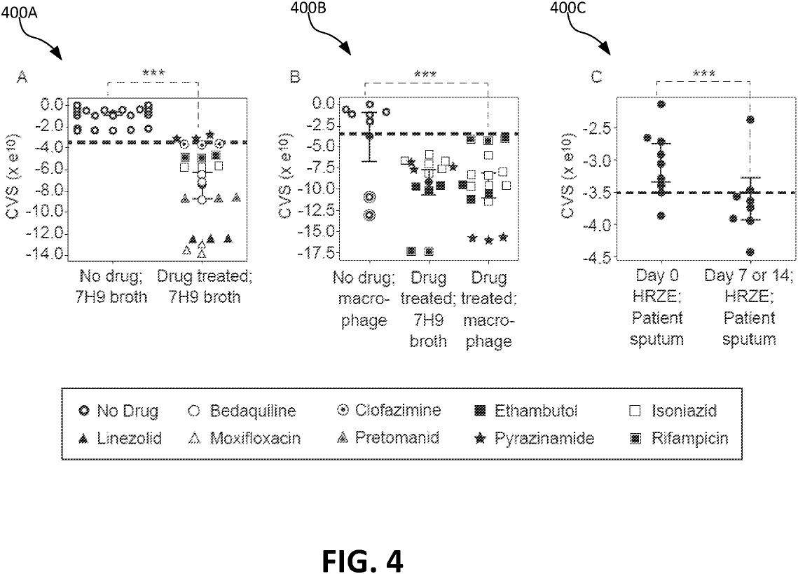

FIG. 4 shows classifier-generated CVSs for transcriptomes of Mycobacterium tuberculosis sourced from broth culture, macrophage infection and subject sputum. Graph 400A shows CVSs for transcriptomes of Mycobacterium tuberculosis cultures grown in 7H9-rich media with or without drug treatment for 72 hours. Graph 400B shows CVSs for transcriptomes of Mycobacterium tuberculosis cultured in 7H9 broth with drug treatment for 24 hours and macrophage with or without drug treatment for 24 hours. Circles with borders indicate transcriptomes from interferon gamma activated macrophages with lipopolysaccharide treatment. Graph 400C shows CVSs for transcriptomes of Mycobacterium tuberculosis in subject sputum collected at the start and end of 7 or 14 day chemotherapy with HRZE; isoniazid (H), rifampicin (R), pyrazinamide (Z), and ethambutol (E). The dashed line in the graphs 400A-C is the cell viability threshold (-3.5e10), below which the samples are considered to be non-viable. Dot and error bars indicate the mean and standard deviation away from the mean. Statistical significance (dashed line that extends out of the graphs 400A-C) was calculated as p-value with Student’s T-test. * * *: p-value < 0.001.

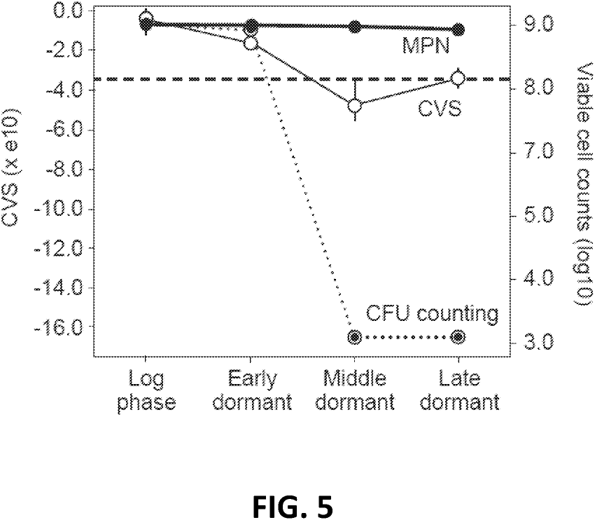

FIG. 5 shows a comparison of CVS from a classifier with bacteriological assays determined by most probable number (MPN) assay and colony forming unit (CFU) enumeration from heterogeneous K+ starved cultures of Mycobacterium tuberculosis transcriptomes. The Mycobacterium tuberculosis transcriptomes for drug response assayer (DRonA) prediction and viable cell counts according to MPN and CFU counting assays were obtained from GEO accession number GSE66408. Log phase is the exponentially growing cultures of Mycobacterium tuberculosis collected prior to K+ starvation, and early, middle, and late dormant are the rifampicin (5 mg/mL)-treated cultures collected after 10, 20, and 30 days of K+ starvation. The filled, unfilled, and outlined dots indicate mean, and error bars indicate standard deviation from the mean. The dashed line is the CVS threshold (-3.5e10) from DRonA and indicates loss of cell viability.

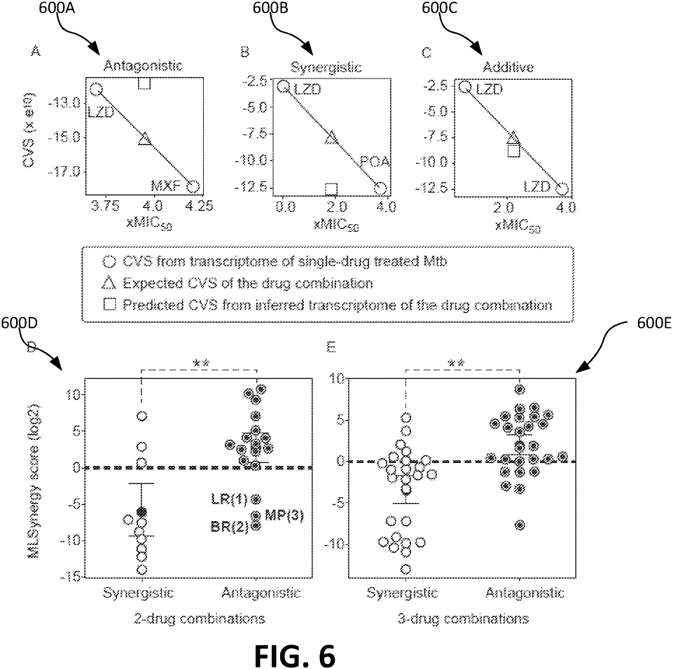

FIG. 6 shows MLSynergy prediction of drug interaction. Examples of the relationship between expected CVS and predicted CVS for antagonistic interactions are shown in graph 600A, for synergistic interactions are shown in graph 600B, and for additive drug combinations are shown in graph 600C. The expected CVS (triangle) was calculated as the average of DRonA-generated CVSs for experimentally measured transcriptomes from single-drug treated Mycobacterium tuberculosis. The drugs include linezolid (LZD), pyrazinoic acid (POA), and moxifloxacin (MXF). Graph 600D shows MLSynergy classification of experimentally validated synergistic and antagonistic two-drug combinations. Drug combinations: (1) linezolid and rifampicin (LR), (2) bedaquiline and pretomanid (BP), and (3) moxifloxacin and pretomanid (MP) were classified as synergistic by MLSynergy. Graph 600E shows MLSynergy classification of experimentally validated synergistic and antagonistic 3-drug combinations. Dot and error bars indicate the mean and standard deviation away from the mean. Statistical significance (dashed line) was calculated as p-value with Student’s T-test. **: p-value < 0.01.

### Collection and Curation of Mycobacterium Gene Expression Omnibus Dataset for Training of DRonA

GEOParser was developed to download transcript profiles and metadata of drug-treated and untreated samples of Mtb-H37Rv from Gene Expression Omnibus (GEO). GEOparser collected median spot intensity from microarray samples and Reads Per Kilobase of transcript, per Million mapped reads (RPKM) from RNA-seq samples. The compendium dataset was curated by removing samples with low coverage (e.g., samples with <70% of annotated Mycobacterium tuberculosis genes). The curated dataset was normalized by rank normalization.

### Manual Labeling of Mycobacterium Tuberculosis Transcriptomes

Using the metadata collected by GEOParser, transcriptomes were labelled as “viable” if the sample description stated that Mycobacterium tuberculosis cultures were grown in optimal growth conditions (mid-log phase of growth in 7H9-rich media, incubated at 37° C. with aeration) and “non-viable” if the sample description stated that Mycobacterium tuberculosis cultures were treated with more than 1x MIC50 drug for more than 12 hours. The remaining transcriptomes were labeled as “unclassified”. Labels were saved as a comma separated value (.csv) file.

### Training and Running DRonA

Rank, normalized transcriptomes along with the labels were provided to a single class support vector machine (SC-SVM) classifier to start the iterative training of DRonA, which is a machine-learning algorithm of the classifier. Each iteration consisted of the following steps: (1) a SC-SVM was trained on the training set (e.g., transcriptomes labelled as “viable”); (2) the accuracy of the trained SC-SVM was calculated with Equation 1 using the test set (e.g., transcriptomes labelled as “non-viable” initially and ones classified as “viable” through the iteration process);

$$\begin{matrix}A c c u r a c y = \\ \frac{T r u e p o s i t i v e + T r u e n e g a t i v e s}{T r u e p o s i t i v e + T r u e n e g a t i v e s + F a l s e p o s i t i v e + F a l s e n e g a t i v e}\end{matrix} \qquad \text{(Equation 1)}$$

(3) assessment of the accuracy; (4) using the trained SC-SVM from (1), viability was predicted in transcriptomes labelled as “unclassified”; and (5) newly predicted viable transcriptomes from the unclassified set were moved to the training set. The iterative process was stopped when the accuracy of the classifier dropped below an accuracy threshold (85%) or when no new transcriptomes from the unclassified set were found to be viable. The cell viability scores (CVS) were calculated for samples as the weighted sum of gene expression ranks using the trained SC-SVM. CVSs were normalized by subtracting the score of a sample with the maximum score observed in that experiment.

### Inference of Multi-Drug Transcriptomes (Triangulation)

Transcriptomes of the Mycobacterium tuberculosis cultures treated with multi-drug combinations at effective doses were predicted by triangulation with the single-drug treated transcriptomes and untreated control. Triangulation was called through ‘triangulate’ function in the MLSynergy algorithm, is another machine-learning algorithm of the classifier that collects transcriptomes of the drugs in combination (each profiled as single-drug) and untreated control and averages them with geometric mean. The inferred multi-drug transcriptomes were then returned to DRonA for CVS determination.

### Calculation of MLSynergy Scores for Drug Combinations

Expected CVSs were obtained from DRonA with the transcriptomes of the single-drug treatments that make up the drug combination and “expected CVS” was calculated by averaging the CVSs of single-drug treatments. The “predicted CVS” was obtained from DRonA with the inferred transcriptome of the drug combination. MLSynergy scores were calculated as the ratio of expected CVS and predicted CVS. Further, MLSynergy scores were log normalized (base 2) in reference to the average of MLSynergy scores of same drug combinations that are considered to be additive in nature.

### Comparison of INDIGO-MTB and MLSynergy Predictions

Two INDIGO (Ma et al., 2019) were retrained with default parameters. Model-1 was trained with the complete dataset (202 combinations and 46 drugs) and Model-2 was trained with partial dataset (98 combinations and 40 drugs) which was obtained after excluding combinations with bedaquiline, clofazimine, linezolid, moxifloxacin, pretomanid and pyrazinamide. Both models were tested on the combinations given in Table 4. Transcriptomes provided in Ma et al. were used as input for the INDIGO models. Transcriptomes generated in this study (summarized in Table 3) were used as input for the MLSynergy.

TABLE 4

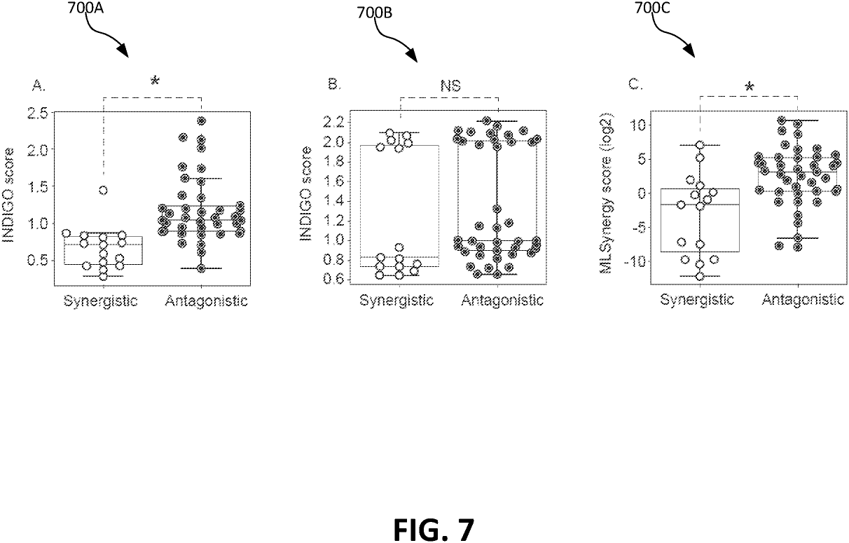

MLSynergy and INDIGO scores for 2- and 3-drug combinations, related to graph 600D and FIG. 7

| Drug 1 | Drug 2 | Drug 3 | Interaction type (DiaMOND interpreted) | MLSynergyscore | Interactive type MLSnergy interpreted | INDIGO score (Model 1) | INDIGO score (Model 2) |
|---|---|---|---|---|---|---|---|
| BDQ | CFZ | | Synergy | 7.08 | Synergy | 0.28 | 2.10 |
| BDQ | INH | | Antagony | 7.12 | Synergy | 1.20 | 2.22 |
| BDQ | LZD | | Antagony | 10.17 | Synergy | 1.20 | 2.09 |
| BDQ | MXF | | Antagony | 10.72 | Synergy | 2.38 | 2.16 |
| BDQ | PA824 | | Antagony | 2.74 | Synergy | 1.04 | 2.12 |
| BDQ | POA | | Synergy | 2.91 | Synergy | | |
| BDQ | RIF | | Antagony | 4.11 | Synergy | 1.76 | 2.12 |
| CFZ | INH | | Antagony | -6.57 | Synergy | 0.39 | 0.95 |
| CFZ | LZD | | Antagony | 0.98 | Synergy | 0.98 | 0.66 |
| CFZ | MXF | | Antagony | 3.13 | Synergy | 1.74 | 0.85 |
| CFZ | PA824 | | Synergy | -12.18 | Synergy | 1.44 | 0.83 |
| CFZ | POA | | Synergy | -11.05 | Synergy | | |
| CFZ | RIF | | Synergy | -7.47 | Synergy | 0.47 | 0.65 |
| INH | LZD | | Antagony | 3.28 | Synergy | 0.86 | 0.99 |
| INH | MXF | | Antagony | 5.19 | Synergy | 2.01 | 1.18 |
| INH | PA824 | | Antagony | -7.91 | Synergy | 1.00 | 1.13 |
| INH | POA | | Synergy | -7.10 | Synergy | | |
| INH | RIF | | Antagony | -4.32 | Synergy | 1.37 | 1.32 |
| LZD | MXF | | Antagony | 9.27 | Synergy | 1.56 | 1.00 |
| LZD | PA824 | | Antagony | 0.35 | Synergy | 1.61 | 0.96 |
| LZD | POA | | Synergy | 0.69 | Synergy | | |
| LZD | RIF | | Antagony | 2.23 | Synergy | 0.95 | 0.72 |
| MXF | PA824 | | Antagony | 2.49 | Synergy | 2.16 | 1.15 |
| MXF | RIF | | Antagony | 4.04 | Synergy | 2.12 | 0.99 |
| PA824 | POA | | Synergy | -13.87 | Synergy | | |
| PA824 | RIF | | Synergy | -9.74 | Synergy | 0.42 | 0.93 |
| POA | RIF | | Synergy | -8.72 | Synergy | | |
| BDQ | CFZ | INH | Synergy | 2.01 | Synergy | 0.38 | 1.99 |
| BDQ | CFZ | LZD | Synergy | 5.29 | Synergy | 0.81 | 1.94 |
| BDQ | CFZ | MXF | Antagony | 6.38 | Synergy | 0.89 | 2.00 |
| BDQ | CFZ | PA824 | Synergy | -0.22 | Synergy | 0.86 | 2.02 |
| BDQ | CFZ | POA | Synergy | 0.05 | Synergy | | |
| BDQ | CFZ | RIF | Synergy | 1.17 | Synergy | 0.42 | 1.95 |
| BDQ | INH | LZD | Antagony | 5.42 | Synergy | 0.85 | 1.95 |

FIG. 7 shows a comparison of INDIGO28 models with MLSynergy in predicting interaction of 2- and 3-drug combinations, related to graph 600D. Graph 700A shows predictions from INDIGO model that was trained on 202 drug combinations with 46 drugs (Model-1). Graph 700B shows predictions from INDIGO model that was trained on 98 drug combination with 40 drugs (Model-2); specifically combinations with bedaquiline, clofazimine, linezolid, moxifloxacin, pretomanid and pyrazinamide were excluded from training. Graph 700C shows predictions from MLSynergy model with same drug combinations as graph 700B. Drugs were validated as synergistic and antagonistic from DiaMOND assay. Statistical significance (black dashed line) was calculated as p-value with Student’s T-test. *: p-value < 0.05.

### Quantification and Statistical Analysis

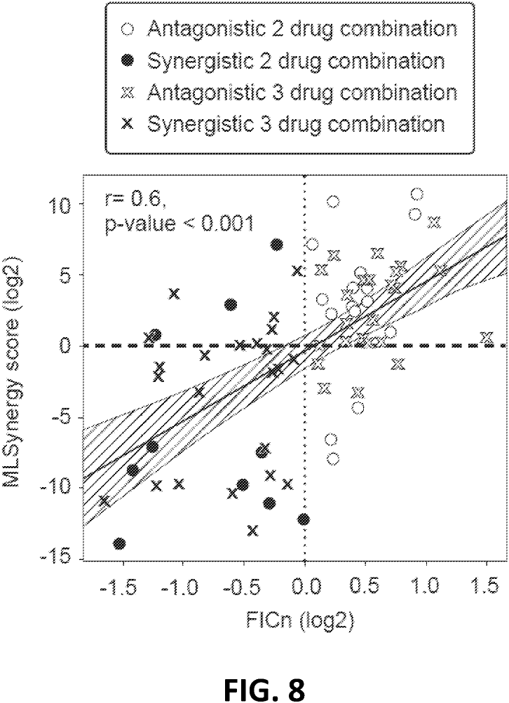

All statistical analysis reported were performed with SciPy package in Python. The p-value from the Student’s t test, sample mean and SEM were used as indicated in FIGS. 3-8. Statistically non-significant (NS) were considered with p-value greater than 0.05 and other qualifying p-values were indicated accordingly * less than 0.05, ** less than 0.01, and *** less than 0.001. The correlations reported in FIGS. 3 and 8 were calculated as the average correlation coefficient, r, from 100 iterations performed with 70% randomly selected data, r and p-values were reported in the figures.

FIG. 8 shows the correlation between MLSynergy score and FICs for two- and three-drug combinations, related to graph 600D. The solid line denotes the Pearson’s correlation between CVS and relative CFU. Significance was calculated as the average correlation coefficient, r, from 100 iterations performed with 70% randomly selected data. The dotted line and dashed line are the FICs and MLSynergy scores, respectively, that separate synergistic combinations from the antagonistic combinations.

### Data and Code Availability

The raw sequencing data have been deposited in GEO with accession number GSE165673. Information is also listed in Tables 5-7.

TABLE 5

Chemicals, peptides, and recombinant proteins resources

| REAGENT or RESOURCE | SOURCE | IDENTIFIER |
|---|---|---|
| Middlebrook 7H9 Broth Base (7H9 broth) | Millipore-Sigma | M0178-500G |
| Middlebrook 7H10 Agar Base (7H10 agar) | Millipore-Sigma | M0303 |
| Bedaquiline | Millipore-Sigma | 843663-66-1 |
| Clofazimine | Millipore-Sigma | 2030-63-9 |
| Isoniazid | Millipore-Sigma | 54-85-3 |
| Linezolid | Millipore-Sigma | 165800-03-3 |
| Moxifloxacin hydrocholoride | Millipore-Sigma | 186826-86-8 |
| Pretomanid | Millipore-Sigma | 187235-37-6 |
| Pyrazinecarboxylic acid (Pyrazinoic acid) | Millipore-Sigma | 98-97-5 |
| Rifampicin | Millipore-Sigma | 13292-46-1 |
| SuperScript II Reverse Transcriptase | ThermoFisher | 18064014 |

TABLE 6

Commercial assays, deposited data, and experimental models: organisms/strains

| REAGENT or RESOURCE | SOURCE | IDENTIFIER |
|---|---|---|
| Commercial assays | | |
| Ribo-Zero Bacteria rRNA Removal Kit | Illumina | 20040526 |
| TruSeq Stranded mRNA HT library preparation kit | Illumina | 20020595 |
| Deposited data | | |
| Transcriptomes from single drug treated Mycobacterium tuberculosis | This study | GEO: GSE165673 |
| Mycobacterium tuberculosis Transcriptome compendium for training of DRonA | This study | GitHub: baliga-lab/DRonA MLSynergy |
| Trained DRonA model (MTB_2020) used in this work | This study | GitHub: baliga-lab/DRonA MLSynergy |
| Experimental models: Organisms/strains | | |
| Mycobacterium tuberculosis: H37Rv | ATCC | 27294 |

TABLE 7

Software and algorithms

| | | |
|---|---|---|
| RESOURCE | SOURCE | IDENTIFIER |
| Python | https://www.python.org/ | N/A |
| SciPy | https://www.scipy.org/ | N/A |
| GEOparser | This study | Zenodo: https://doi.org/10.5281/zenodo.5598251, GitHub: baliga-lab/DRonA MLSynergy |
| DRonA | This study | Zenodo: https://doi.org/10.5281/zenodo.5598251, GitHub: baliga-lab/DRonA MLSynergy |
| MLSynergy | This study | Zenodo: https://doi.org/10.5281/zenodo.5598251, GitHub: baliga-lab/DRonA MLSynergy |
| Google Colab notebook for DRonA and MLSynergy | This study | Zenodo: https://doi.org/10.5281/zenodo.5598725, GitHub: baliga-lab/Google-colab-notebooks/ blob/master/DRonA MLSYnergy.ipynb |

### Drug Response Assayer (DRonA) Detects Signatures for Loss of Viability Within Transcriptomes of Mycobacterium tuberculosis Irrespective of Mechanism of Killing

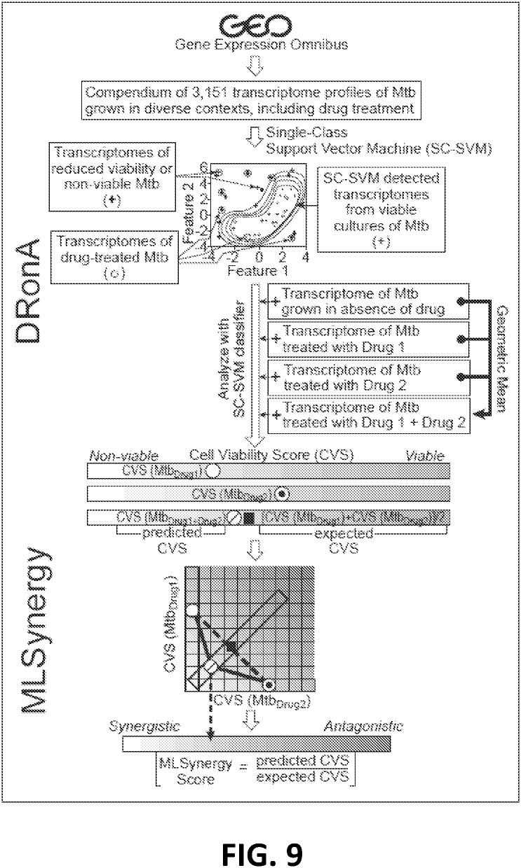

To investigate whether Mycobacterium tuberculosis viability can be deciphered from its transcriptome state, the study sought to define a classifier that could accurately identify transcriptomes of viable Mycobacterium tuberculosis. It was hypothesized that the degree of deviation of a transcriptome from the boundary defined by the classifier would indicate the loss of viability of Mycobacterium tuberculosis cells. Further, it was hypothesized that the loss of viability would be agnostic of the inhibitory effect, making it possible to predict drug-mediated killing, irrespective of the mechanism of action (FIG. 9). While there are various classification techniques (e.g., artificial neural networks, decision trees, Bayesian classifiers), the support vector machine (SVM) algorithm is a technique for optimizing the expected solution (e.g., identifying a signature of viable states of Mycobacterium tuberculosis) with limited datasets. Moreover, classification based on SVM offers potential for feature analysis to identify specific genes whose expression levels are diagnostic of the viability state of Mycobacterium tuberculosis. Therefore, a single-class support vector machine (SC-SVM) was trained using a compendium of 3,151 transcriptomes of Mycobacterium tuberculosis grown in diverse conditions to accurately identify the transcriptomes that belong to “viable” states of Mycobacterium tuberculosis.

Referring to FIG. 9, an overview schematic of DRonA/MLSynergy framework is shown. DRonA is a SC-SVM that was trained on transcriptomes from viable Mycobacterium tuberculosis cultures. DRonA was trained through an iterative process to define a region in the hyperplane that classifies transcriptomes from Mycobacterium tuberculosis grown in varying growth conditions as viable and distinguishes them from non-viable transcriptomes (i.e., drug treated at greater than MIC50 concentration). DRonA takes transcriptomes as input and outputs a CVS, which is the empirical distance from the viable class and indicative of efficacious drug treatment. Using an inferred transcriptome of a drug combination from single-drug transcriptomes, MLSynergy predicts the outcome of the drug interaction.

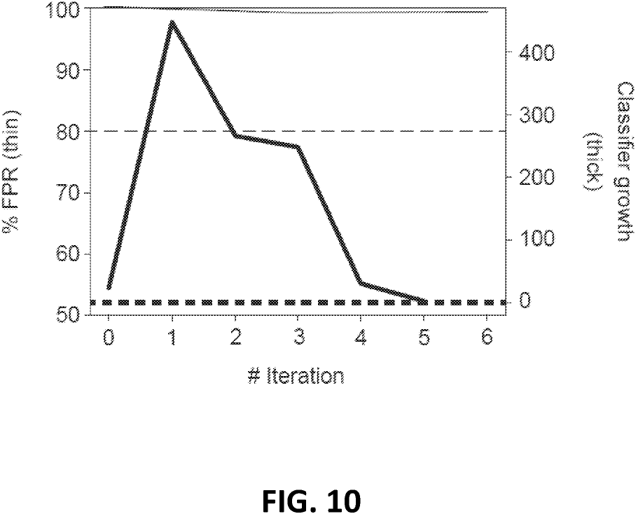

The compendium of 3,151 transcriptomes was compiled from 173 studies available in the Gene Expression Omnibus (GEO). These studies used microarray and RNA sequencing (RNA-seq) to assess gene expression changes in Mycobacterium tuberculosis from various growth medium compositions, culture conditions, and drug treatment. Batch effects and platform-specific bias across the transcriptome profiles were corrected with rank normalization, and each profile was labeled as “viable”, “non-viable”, or “unclassified” by manual inspection of the associated metadata. Specifically, 24 transcriptomes of Mycobacterium tuberculosis cultured in optimal growth conditions (mid-log phase of growth in 7H9 nutrient-rich media, incubated at 37° C. with aeration) were labeled as “viable” and 193 transcriptomes of Mycobacterium tuberculosis cultures treated with 17 different drugs at greater than 13 MIC50 for greater than 12 hours were labeled as “non-viable”. The remaining 2,319 transcriptomes were labeled as “unclassified”. The labeled transcriptome compendium was used for SC-SVM training, which was performed to broaden the classifier-defined boundary of viability by iteratively including transcriptomes from the “unclassified” set that were from viable Mycobacterium tuberculosis adapted to non-lethal, sub-optimal growth conditions. The classifier was iteratively trained on the “viable” set until addition of transcriptomes from the “unclassified” set caused a drop in its performance in accurately classifying viable and non-viable transcriptomes (FIG. 10). The final classifier was trained on 994 transcriptomes of Mycobacterium tuberculosis from diverse growth conditions, including log phase, vehicular control samples, nutrient starvation, low pH, hypoxia, and intracellular growth. As such, the SC-SVM classifier identified Mycobacterium tuberculosis transcriptomes from slow-growing (e.g., dormancy inducing), but viable conditions. In contrast, the excluded transcriptomes (total 1,940) were from stressful conditions (e.g., drug treated, heat treated, amino acid starved) and lethal genetic perturbations (e.g., phoP, espR, mihF mutants) that reduced cell viability in Mycobacterium tuberculosis cultures.

Referring to FIG. 10, iterative training of DRonA is shown. The graph shows changes in accuracy, measured as percentage false positive rate (% FPR in thin line) and number of non-classified transcriptomes added to the viable training (classifier growth in a thick line) at each iteration. The % FPR was calculated as FP/N, where FP is the number of drug treated non-viable transcriptomes (from test set) that were classified as viable and N is the total number of non-viable transcriptomes (from test set). Thin and thick dashed line show the threshold for % FPR and classifier growth below which the iterative training was programmed to stop. The classifier growth dropped below threshold at the fifth iteration and was halted.

The linear SC-SVM classifier, named drug response assayer (DRonA), took as input transcriptomes of Mycobacterium tuberculosis to calculate a CVS. The calculated CVS was proportional to the deviation of a given transcriptome from the lower limit of the classifier-defined viable transcriptome space. This lower limit was set as the cell viability threshold (e.g., cell viability threshold of -3.5e¹⁰), below which a CVS indicates a transcriptome signature of nonviable Mycobacterium tuberculosis. Using an independent compendium of 72 transcriptomes generated for this study (Table 3), it was ascertained that the CVS scoring scheme of DRonA accurately classified as “viable” (e.g., with a CVS greater than -3.Se¹⁰) all 27 transcriptomes of Mycobacterium tuberculosis grown in 7H9 medium in the absence of drugs. By contrast, DRonA predicted loss of viability (e.g., CVS less than -3.Se¹⁰) from transcriptomes of Mycobacterium tuberculosis cultures treated for 72 hours in 7H9 growth medium with each of the seven frontline tuberculosis drugs at R MIC50 concentration (p value < 0.001, graph 400A). As expected, pyrazinamide treatment at 3.0 mg/mL was not predicted to reduce the viability of Mycobacterium tuberculosis. Next, the performance of DRonA in predicting Mycobacterium tuberculosis viability within an intracellular host context was tested, using as input 39 transcriptomes of Mycobacterium tuberculosis from naive, lipopolysaccharide (LPS)-activated, and drug-treated infected macrophages of J774A.1 lineage (Table 3). Again, DRonA correctly classified the transcriptomes from untreated Mycobacterium tuberculosis as viable and the drug-treated transcriptomes as non-viable (graph 400B). Moreover, DRonA detected the known increase in the intracellular efficacy of pyrazinamide and also the decreased efficacy of rifampicin in killing Mycobacterium tuberculosis within macrophages. DRonA also detected a loss in the viability of Mycobacterium tuberculosis within interferon-gamma-activated macrophages upon LPS treatment. Together this demonstrates that DRonA was able to identify non-viable transcriptomes, irrespective of the context and underlying mechanism of killing (e.g., whether immune or drug induced). Finally, the performance of DRonA in predicting drug response within tuberculosis subjects was tested, using as input 16 transcriptomes of Mycobacterium tuberculosis from the sputum of eight subjects at the start of and after 7 or 14 days of successful tuberculosis treatment with isoniazid (H), rifampicin (R), pyrazinamide (Z), and ethambutol (E). DRonA efficiently differentiated cell viability from the Mycobacterium tuberculosis transcriptomes collected from subjects on day 0 from transcriptomes collected on day 7 or 14 of drug treatment (p value < 0.01) (graph 400C), demonstrating that DRonA can detect drug treatment response from bacterial RNA in subject sputum.

### DRonA Estimation of the Decline in CFUs Upon Drug Treatment

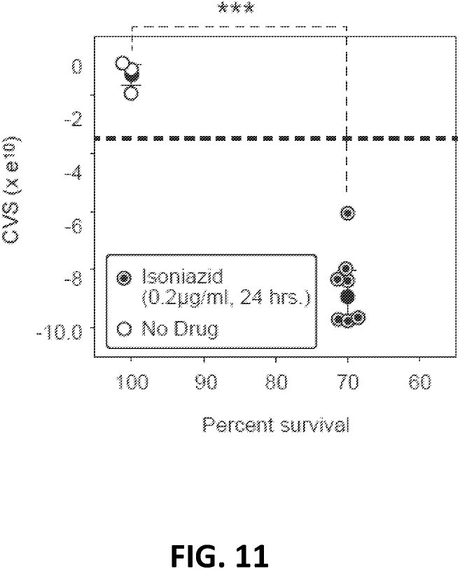

The study involved testing whether the CVS was proportional to the magnitude of drug effects based on CFU assessment. DRonA-generated CVSs were compared with the relative CFUs observed after Mycobacterium tuberculosis was treated for 24 and 72 hours with seven frontline tuberculosis drugs at various concentrations and conditions (Tables 1 and 2). The CVS scores calculated from transcriptomes of both untreated Mycobacterium tuberculosis cultures and those treated with drugs at less than MIC50 concentrations were higher than the viability threshold. Although, the inferred CVS from cultures treated with less than MIC50 drug was less than the CVS of untreated cultures (difference in average = -3.53e¹⁰, p value < 0.01), indicating a moderate loss of viability. In contrast, the CVS scores calculated from transcriptomes of Mycobacterium tuberculosis cultures treated with RMIC50 concentration of drug were consistently below the viability threshold. Furthermore, for both Mycobacterium tuberculosis grown in 7H9 medium and within macrophages, the reduction in CVS was proportional to the decrease in CFU for most drugs (FIG. 3 and FIG. 11), with the exception of bedaquiline. It is known that bedaquiline kills Mycobacterium tuberculosis relatively slowly compared with other frontline drugs and could explain the discord between CFU and CVS within 72 hours of treatment. Despite the slow bactericidal activity of bedaquiline, its lethal effect was captured in transcriptome changes at a significantly earlier time point, and a significant correlation between relative CFUs and CVS across all drug treatments was observed (r = -0.9, p value < 0.001).

FIG. 11 shows that CVS accurately predicts bactericidal effects of isoniazid (INH) on Mycobacterium tuberculosis in intracellular environment, related to FIG. 3. CVS was calculated using DRonA analysis of Mycobacterium tuberculosis transcriptomes from infected macrophages. CVS was correlated with percent survival generated from CFU data for intracellular Mycobacterium tuberculosis with and without 0.2 µg/ml INH treatment. The thick dashed line is the cell viability threshold (-3.5e10), below which the samples are considered to be non-viable. Black dot and error bars indicate the mean and standard deviation away from the mean. Statistical significance (thin dashed line) was calculated as p-value with Student’s T-test. ***: p-value < 0.001.

A disadvantage of performing drug response assessment via CFU counting is the limitation that it only measures culturable bacteria. Mycobacterium tuberculosis from in vivo models of latent tuberculosis infection are non-culturable and require resuscitation-promoting factors or conditions to resume growth. Thus, CVS scores determined using mRNA signatures represent a comprehensive assay of drug effects on dormant Mycobacterium tuberculosis that are unable to grow on solid medium but retain full potential of recovering to a physiologically active state. To test this hypothesis, the study investigated the accuracy of DRonA in predicting Mycobacterium tuberculosis killing by a moderate concentration of rifampicin (5 mg/mL) in potassium-deficient growth medium. Mycobacterium tuberculosis shifts to a dormant state that is unable to grow on solid medium, but able to recover and proliferate in albumin, dextrose, and sodium chloride (ADC)-supplemented Sauton medium containing potassium. The results demonstrated that CFU counting overestimated rifampicin-treatment-induced killing of the pathogen, as demonstrated by a minimum probable number (MPN) performed in the same context in ADC-supplemented liquid Sauton medium. Notably, similar to MPN results, there was no significant drop in CVS, demonstrating that DRonA accurately predicted the overall drug response in cultures that consist of non-culturable Mycobacterium tuberculosis (FIG. 5).

### MLSynergy Prediction of Synergistic and Antagonistic Drug Combinations from Transcriptomes of Single-Drug-Treated Mycobacterium Tberculosis

Given that DRonA can detect Mycobacterium tuberculosis’ response to drug treatment from gene expression data, the study investigated if DRonA could be used to accelerate multicomponent drug discovery by predicting the outcome of drug interactions from single-drug-treated transcriptomes. To do this, an approach to infer the transcriptomes of multidrug treatments was developed. Specifically, the transcriptome of multidrug combinations was inferred by triangulation of the respective transcriptomes obtained from single-drug-treated cultures of Mycobacterium tuberculosis and then used the inferred multidrug transcriptome with DRonA to predict the CVS of the multidrug combination (e.g., the “predicted CVS”). Transcriptomes used for prediction of drug interactions were from Mycobacterium tuberculosis treated with single drugs in matched experimental conditions (7H9 medium and 72 hours drug treatment).

Using this method to predict the CVS of multidrug combinations, a parametric method, “MLSynergy”, was developed to predict the interaction outcome of the two- and three-drug combinations. MLSynergy predicts the synergy or antagonism of multidrug combinations based on the Loewe additivity principle by calculating the ratio of predicted CVS to expected CVS, where the “expected CVS” for a drug combination is the average of CVSs from respective single-drug treatments (FIG. 9). For example, the predicted CVS of the antagonistic combination linezolid and moxifloxacin is greater than the expected CVS and lies above the additive plane (graph 600A), whereas the predicted CVS of the synergistic combination linezolid and POA is less than the expected CVS and lies below the additive plane (graph 600B). Finally, the predicted CVS of linezolid with itself is the same as the expected CVS and lies on the additive plane, consistent with the Loewe additivity principle, which states that a drug in combination with itself is additive in interaction (graph 600C). As such, an MLSynergy score less than 1 predicts that the drug interaction is synergistic, and a score greater than 1 indicates an antagonistic drug interaction. MLSynergy scores were calculated for all two- and three-drug combinations of eight frontline drugs (Table 3). The MLSynergy predictions of 26 two-drug and 40 three-drug combinations of the eight frontline drugs were compared with their experimentally determined interaction, quantified by fractional inhibitory concentrations (FICs). This comparative analysis demonstrated that MLSynergy was greater than 90% accurate in predicting synergistic and antagonistic effects of two- and three-drug combinations (graphs 600D-E). Moreover, MLSynergy scores were highly correlated with the FIC values (r = 0.61, p value < 0.001, FIG. 8). Interestingly, three two-drug combinations (identified with text in graph 600D) were predicted as synergistic by MLSynergy, but were determined to be antagonistic by DiaMOND assay. Notably, these combinations were previously determined to be synergistic by other studies, suggesting that the effect of their drug interaction could be highly dependent on the assay method and conditions.

Finally, the ability of MLSynergy to predict condition-dependent drug interactions in Mycobacterium tuberculosis was checked using as input 22 transcriptomes of Mycobacterium tuberculosis from untreated and drug-treated infected macrophages of J774A.1 lineage (Table 3). Drug interaction was predicted for two- and three-drug combinations of isoniazid, rifampicin, and pyrazinamide in both broth culture and macrophages and the MLSynergy predictions were compared with their experimental FIC values, as shown in Table 8. MLSynergy predicted that all the drug combinations are synergistic in 7H9 media and turn antagonistic in macrophage. Similarly, the experimental results found that mostly all the drug combinations (with the exception of isoniazid + rifampicin) are synergistic in broth and antagonistic in macrophage. This demonstrates that MLSynergy is robust to the context in which a drug effect is measured, and it can predict condition-dependent drug interactions.

TABLE 8

MLSynergy scores and FIC50 values of two- and three-drug combinations in broth and macrophage context related to graph 600D

| Drug | | | MLSynergy scores | | FIC₅₀ value (log₂) | |
|---|---|---|---|---|---|---|
| 1 | 2 | 3 | 7H 9 | Macrophage | 7H 9 | Macrophage |
| PZA | RIF | | -8.72 | 2.7 | -1.42 | -0.11 |
| INH | PZA | | -7.1 | 2.0 | -1.26 | 0.42 |
| INH | RIF | | -4.3 | 2.8 | 0.44 | -0.07 |
| INH | PZA | RIF | -7.13 | 2.45 | -5.81 | 0.29 |

MLSynergy predicted and experimentally determined interactions of pyrazinamide (PZA), isoniazid (INH), and rifampicin (RIF) from Mycobacterium tuberculosis growing in 7H9 media or infected J774A.1 macrophages. Drug combinations with MLSynergy score and FIC value (log2) < 0 are considered synergistic and > 0 are considered antagonistic in interaction.

### Discussion

The study supports use of a machine learning framework for drug response prediction in Mycobacterium tuberculosis. DRonA enables efficient prediction of cell viability from transcriptomic signatures of perturbation, including drug treatment. Using DRonA estimates of cell viability from single-drug transcriptomic data, MLSynergy can then predict synergy and antagonism of antitubercular drug combinations. The analysis using DRonA found a strong association between in silico estimates of cell viability following drug treatment and experimentally observed reduction in CFUs. Moreover, the loss of viability captured by DRonA from Mycobacterium tuberculosis transcriptomes of subjects undergoing HRZE treatment supports the clinical utility of the approach. Finally, the study found several synergistic drug combinations, suggesting that the DRonA/MLSynergy framework is a promising tool for the prioritization of new multicomponent drug regimens. While thr predictions of two- and three-drug interactions were validated, the framework is generalizable for higher-order combinations.

The suitability of using the transcriptome as a reflection of Mycobacterium tuberculosis viability was studied by treating Mycobacterium tuberculosis with seven frontline drugs and isolating RNA for transcriptome profiling, while also evaluating cell viability by CFU. The DRonA predicted the CVS of Mycobacterium tuberculosis exposed to bactericidal (e.g., greater than MIC50) concentrations of drugs were below the cell viability threshold, proportional to relative CFU and significantly different from the CVS of untreated Mycobacterium tuberculosis cultured for the same duration as drug treatment. Moreover, DRonA was able to perform effectively with other transcriptomic datasets of Mycobacterium tuberculosis drug treatment, including during macrophage infection and from tuberculosis subjects. The ability of DRonA to accurately predict the consequence of drug treatment in 7H9 medium, within macrophages, and from subject sputum, demonstrates that the definitions of viability in the DRonA model are inclusive of both actively dividing and slow replicating (physiologically adapted) phenotypes of Mycobacterium tuberculosis. Moreover, the accuracy across datasets offers DRonA as a generalizable tool for use across drug response screens and in studies where gene expression was analyzed, but Mycobacterium tuberculosis viability was not measured.

Here, it was shown that DRonA complements bacteriological assays in evaluating treatment response. The decline in CVS corresponded to the decline in the proportions of surviving bacilli upon drug treatment, as measured by the relative CFU counts. Since no culturing is required, DRonA can estimate drug effects faster than conventional bacteriological assays. Additionally, the ability to enrich and amplify RNA may allow DRonA to be used with samples where bacterial cell numbers are low. The high sensitivity and the autonomy from culturing makes DRonA especially promising to evaluate the efficacy of treatment regimens on dormant non-culturable Mycobacterium tuberculosis that are associated with latent infection in humans.

Using DRonA-predicted viability scores, MLSynergy accurately predicted synergy and antagonism for two- and three-drug combinations. This performance compares with INDIGO-MTB, an existing strategy that quantifies synergistic and antagonistic drug regimens using transcriptomes of Mycobacterium tuberculosis treated with individual drugs, but only with drugs with known drug-drug interactions. INDIGO-MTB requires known drug-drug interactions to learn patterns and identify combinations most likely to be synergistic. In contrast, the DRonA/MLSynergy platform is based on gene signatures of cell viability and does not require any input data related to drug combinations. Comparing the accuracy for drugs without prior drug interaction information, MLSynergy significantly outperforms INDIGO-MTB (p value > 0.05, FIG. 9). As such, the models can be more easily applied (e.g., no re-training required) to predict drug interaction for new drugs and conditions.

Second, the DRonA/MLSynergy platform requires transcriptome profiling of Mycobacterium tuberculosis drug treatment to predict drug interactions. However, predicting drug interactions using transcriptome analysis with DRonA/MLSynergy is cheaper and faster, as compared with bacteriological assays. Evaluating drug interactions with bacteriological assays requires a significantly larger number of experiments, which increases exponentially with every new drug and for testing higher-order (e.g., three-drug) interactions. For example, to evaluate all possible two-drug interactions between 10 drugs (e.g., 45 combinations), a checkerboard or DiaMOND assay would require a minimum of 55 experiments, whereas MLSynergy would require just 10 experiments to generate transcriptomes of Mycobacterium tuberculosis in response to treatment with each of the 10 drugs. For three-drug combinations, checkerboard or DiaMOND assay requirement increases to 120 drug dose titration experiments, whereas requirements for MLSynergy remains the same (e.g., 10 experiments). Furthermore, technological advancements are making it faster and cheaper to profile the transcriptome of Mycobacterium tuberculosis directly from subject samples, which could potentially extend the utility of DRonA in rapid point-of-care devices for evaluating the effectiveness of drug treatment in tuberculosis subjects.

Drug response prediction with machine learning models is an important area of current research, particularly for a slow-growing pathogen, and the results highlight the practicality of using transcriptome signatures to address major bottlenecks in the drug discovery process. The ability to detect changes in cell viability and predict drug interaction using just transcriptome profiles could substantially accelerate tuberculosis drug discovery efforts. Recent studies have demonstrated that efficacy of the same drug combination can vary significantly between broth conditions and animal models. DRonA and MLSynergy could be valuable for prioritizing drug combinations that are likely to be effective in animal models, given the challenges in performing high-throughput drug assays in mouse models and non-human primates. Finally, the DronA/MLSynergy framework can be easily extended to predict other genotypes and phenotypes of Mycobacterium tuberculosis associated with a gain in drug resistance (e.g., metabolic states and cell wall composition), which could further improve treatment response prediction and clinical outcomes.

### Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed:

1. A computer-implemented method comprising:
(a) accessing a disease agent transcriptome of a disease agent;
(b) generating a disease agent viability score by applying a classifier to the disease agent transcriptome, the classifier defining a universal transcriptome signature for a viability of the disease agent in a plurality of different host-relevant contexts;
(c) generating a viability state of the disease agent by determining a deviation of the disease agent viability score from a viability threshold of the universal transcriptome signature for viability;
(d) determining a treatment recommendation based on the viability state of the disease agent; and
(e) outputting the treatment recommendation.

2. The computer-implemented method of claim 1, wherein the classifier was trained using a training data set comprising a plurality of viable disease agent transcriptomes, and wherein the classifier was tested on a testing data set comprising a first set of untreated disease agent transcriptomes and a second set of treated disease agent transcriptomes, the training data set and the testing data set derived from the disease agent being grown under the plurality of different host-relevant contexts with drug treatment and without drug treatment to define the universal transcriptome signature for viability.

3. The computer-implemented method of claim 1, wherein the viability threshold is set as a lower limit of a viable transcriptome space defined by the classifier.

4. The computer-implemented method of claim 1, wherein the classifier is a single-class support vector machine.

5. The computer-implemented method of claim 1, wherein the disease agent viability score is a weighted sum of a plurality gene expression ranks generated by the classifier and rank normalized.

6. The computer-implemented method of claim 1, wherein the disease agent is a cell, and the disease agent transcriptome is obtainable from the cell.

7. The computer-implemented method of claim 1, wherein the disease agent is Mycobacterium tuberculosis and a host of the disease agent is a mammal.

8. The computer-implemented method of claim 1, wherein the disease agent transcriptome comprises a subset of mRNA transcripts produced by primer-directed amplification, the subset of mRNA transcripts comprising one or more weighted features selected by bootstrapping and rank ordering based on weights determined by the primer-directed amplification.

9. The computer-implemented method of claim 8, wherein the primer-directed amplification is reverse transcription loop-mediated isothermal amplification (LAMP).

10. The computer-implemented method of claim 1, wherein determining the treatment recommendation comprises:
comparing the viability state of the disease agent to one or more single-drug treatment viability states of the disease agent, the one or more single-drug treatment viability states produced by: (i) generating one or more single-drug treatment viability scores by an application of the classifier to a plurality of single-drug treatment transcriptomes of the disease agent grown under a plurality of single-drug treatment conditions, and (ii) generating the one or more single-drug treatment viability states by a determination of another deviation of the one or more single-drug treatment viability scores from the viability threshold of the universal transcriptome signature for viability.

11. The computer-implemented method of claim 10, wherein determining the treatment recommendation further comprises:
comparing the viability state of the disease agent and the one or more single-drug treatment viability states of the disease agent with a multi-drug viability state, the multi-drug viability state imputed by an application of the classifier to an average of a plurality of disease agent transcriptomes and one or more single drug treatment transcriptomes.

12. The computer-implemented method of claim 11, wherein the average is a geometric mean.

13. The computer-implemented method of claim 1, wherein determining the treatment recommendation comprises evaluating an efficacy of a drug treatment for the disease agent.

14. The computer-implemented method of claim 1, further comprising:
facilitating the treatment recommendation for a host of the disease agent.

15. A computer-program product tangibly embodied in a non-transitory machine-readable storage medium, including instructions configured to cause one or more data processors to perform a set of actions including:
(a) accessing a disease agent transcriptome of a disease agent;
(b) generating a disease agent viability score by applying a classifier to the disease agent transcriptome, the classifier defining a universal transcriptome signature for viability of the disease agent in a plurality of different host-relevant contexts;
(c) generating a viability state of the disease agent by determining a deviation of the disease agent viability score from a viability threshold of the universal transcriptome signature;
(d) determining a treatment recommendation for the disease agent based on the viability state of the disease agent; and
(e) outputting the treatment recommendation.

16. The computer-program product of claim 15, wherein determining the treatment recommendation comprises:
comparing the viability state of the disease agent to one or more single-drug treatment viability states of the disease agent, the one or more single-drug treatment viability states produced by a process comprising an application of the classifier to a plurality of single-drug treatment transcriptomes of the disease agent grown under a plurality of single-drug treatment conditions.

17. The computer-program product of claim 16, wherein determining the treatment recommendation further comprises:
comparing the viability state and the one or more single-drug treatment viability states with a multi-drug treatment viability state.

18. The computer-program product of claim 17, wherein the multi-drug treatment viability state is imputed.

19. The computer-program product of claim 18, wherein the multi-drug treatment viability state is produced by an imputation comprising an application of the classifier to an average of a plurality of disease agent transcriptomes and one or more single-drug treatment transcriptomes.

20. A system comprising:
a microfluidic device for receiving a sample of a host subject and producing disease agent transcriptome data of a disease agent from the sample;
one or more data processors; and
a non-transitory computer readable storage medium containing instructions which, when executed on the one or more data processors, cause the one or more data processors to perform a set of actions including:
(a) accessing a disease agent transcriptome of the disease agent;
(b) generating a disease agent viability score by applying a classifier to the disease agent transcriptome, the classifier defining a universal transcriptome signature for viability of the disease agent in a plurality of different host-relevant contexts;
(c) generating a viability state of the disease agent by determining a deviation of the disease agent viability score from a viability threshold of the universal transcriptome signature;
(d) determining a treatment recommendation for the disease agent based on the viability state of the disease agent; and
(e) outputting the treatment recommendation.
